(19) United States
(12) Patent Application Publication
(10) Pub. No.: US 2022/0344053 A1
(43) Pub. Date: Oct. 27, 2022

(54) METHODS FOR EVALUATING NEUROCOGNITIVE DISEASE PROGRESSION AND THERAPIES USING SURROGATE OUTCOME MEASURES

(71) Applicants: Melissa Jane Hogan, Thompson's Station, TN (US); Kimberly Ann Stephens, Knoxville, TN (US)

(72) Inventors: Melissa Jane Hogan, Thompson's Station, TN (US); Kimberly Ann Stephens, Knoxville, TN (US)

(21) Appl. No.: 17/634,863

(22) PCT Filed: Aug. 12, 2020
(86) PCT No.: PCT/US2020/046041

§ 371 Date: Feb. 11, 2022

Related U.S. Application Data
(60) Provisional application No. 62/885,779, filed on Aug. 12, 2019.

(51) Int. Cl. G16H 50/30; G16H 15/00; G16H 50/70

(52) U.S. Cl. CPC G16H 50/30; G16H 15/00; G16H 50/70

(57) ABSTRACT

Disclosed are methods and materials for evaluating neurocognitive function in certain neurodegenerative disorders, such as Mucopolysaccharidosis type II (MPS II), using surrogate measures rather than traditional measures. A survey of nontraditional surrogate measures relating to toileting tasks is described. The survey is useful for evaluating individuals, groups of patients, and therapies.

### CROSS REFERENCE TO RELATED APPLICATION

The present application claims priority to U.S. Provisional Patent Application No. 62/885,779, filed Aug. 12, 2019, the disclosure of which is hereby incorporated by reference in its entirety herein.

### FIELD OF THE INVENTION

The invention relates to methods for assessing neurocognitive function in patients suspected of having or diagnosed with a neurodegenerative disease.

### BACKGROUND

Mucopolysaccharidosis type II (Hunter syndrome, MPS II, OMIM 309900) is a rare, X-linked disease of glycosaminoglycan metabolism that is caused by a deficiency in the lysosomal enzyme iduronate-2-sulfatase (EC 3.1.6.13). In affected patients, glycosaminoglycans accumulate in lysosomes of various tissues and organs and contribute to the pathophysiology of MPS II. The incidence of MPS II is ˜1 in 100,000 male live births but varies considerably between different populations, and cases have been reported in females. Symptoms of MPS II can generally be categorized as somatic, neurocognitive, and neurobehavioral.

In the traditionally described severe or neuronopathic form of MPS II, the onset of disease becomes apparent within 2-4 years of age, and without treatment for the associated neurodegeneration, death generally occurs before adulthood. The burden of caring for a child with MPS II is significant. There is an urgent need for treatments, especially for the neuronopathic form, which affects approximately two out of three patients with MPS II.

Despite the urgent need, identifying effective therapies for MPS II has been challenging, and several initially promising therapies failed during the late stages of clinical trials. As noted specifically in multiple panels at the U.S. Food and Drug Administration, for neurodegenerative diseases such as MPS II, a successful experimental therapy might only affect incremental changes, such as slowing down the progressive neurological decline, stabilizing cognitive abilities at the current level, or even reversing the neurological impact.

So far, however, efforts to quantify incremental changes in neurocognitive function, including decline, stabilization, and improvement, have been mostly unsuccessful due to the lack of sensitivity of current neurocognitive and adaptive measurement techniques. In clinical trials for MPS II and other diseases involving progressive neurological decline, the most common endpoints utilize standard neurocognitive and adaptive measures.

Therefore, there is a need for alternative strategies for assessing the status and progression of neurocognitive function and the decline associated with certain diseases such as MPS II. Effective assessment using novel measures would improve the evaluation of experimental therapies and would facilitate identification of beneficial therapies that may not be recognized using traditional measures.

### BRIEF DESCRIPTION OF THE FIGURES

FIG. 1 shows toileting abilities percentage (TAP) scores by age and treatment status (including trendlines) in a study comparing patients having received intrathecal idursulfase with those having received idursulfase only, as described in Example 2.

FIG. 2 graphically depicts mean scores on the TAS (5-point Likert scale) for each of the 27 Individual toileting abilities by treatment status in the study described in Example 2.

FIG. 3 graphically depicts mean scores on the TAS (5-point Likert scale) for each of the 27 Individual toileting abilities by treatment status for the Older Population in the study described in Example 2.

FIG. 4 graphically depicts mean scores on the TAS (5-point Likert scale) for each of the 27 Individual toileting abilities by treatment status for overall and age-parsed populations in the study described in Example 2.

### DETAILED DESCRIPTION OF THE INVENTION

Disclosed herein are methods of evaluating neurocognitive function using nontraditional surrogate measures in place of traditional neurocognitive or adaptive measures. The methods demonstrate surprising utility for evaluating progression of neurocognitive diseases and for evaluating experimental therapies.

The present invention addresses evidence and arguments that for certain populations, traditional neurocognitive measures do not necessarily reflect meaningful improvement in skills and abilities that are most important to patients and caregivers. Also, those traditional neurocognitive measures may indicate that a clinical therapy is ineffective, or that any perceived effectiveness is not statistically significant, when in fact the therapy provides improvement to skills and abilities that are meaningful and significant to patients and caregivers.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

The following terms, unless otherwise indicated, shall be understood to have the following meanings:

As used herein, the terms “a”, “an”, and “the” can refer to one or more unless specifically noted otherwise.

The use of the term “or” is used to mean “and/or” unless explicitly indicated to refer to alternatives only or the alternatives are mutually exclusive, although the disclosure supports a definition that refers to only alternatives and “and/or.” As used herein “another” can mean at least a second or more.

Throughout this application, the term “about” is used to indicate that a value includes the inherent variation of error for the device, the method being employed to determine the value, or the variation that exists among samples.

The term “therapy” is used herein to refer to any treatment, such as a drug, dietary change, physical or psychological therapy, etc.

All neurodegenerative diseases cause progressive loss of cognitive abilities. Neurocognitive symptoms of MPS-II and other neurodegenerative diseases that primarily affect children frequently cause developmental delay, speech delay or lack of speech development, and progressive loss of cognitive abilities. Neurobehavioral symptoms in children affected by neurodegenerative diseases often include short attention span and high distractibility, impulsivity, heightened activity, sensory seeking behavior, emotional dysregulation, abnormal social interaction, poor sleep, and a reduced sense of danger.

Treating MPS II traditionally focused on palliative care then progressed to therapies addressing the somatic symptoms. The approval of idursulfase (ELAPRASE®) in 2006 in the United States (and thereafter in many other countries) brought reported stabilization and improvement in somatic symptoms. Haematopoietic Stem Cell Transplantation (HSCT) also has been used with varying degrees of success to alleviate somatic symptoms and, in some cases, with reported stability and improvement in cognitive and behavioral profiles as well. But risks of morbidity and mortality remain high.

Experimental therapies currently under exploration for treatment of the neuronopathic symptoms of MPS II include intrathecal enzyme replacement therapy, gene therapy, gene editing, fusion proteins to permit enzymes to cross the blood-brain-barrier from an intravenous infusion (for example, using the insulin receptor or the transferrin receptor), substrate reduction therapy, and chaperone molecules. In particular, intrathecal enzyme replacement therapy (intrathecal idursulfase) showed good biodistribution throughout the central nervous system (CNS) and provided biochemical and pathological efficacy in preclinical studies.

In clinical trials for experimental therapies for MPS II, the most common endpoints utilize standard neurocognitive and adaptive measures. But no neurocognitive measures have been validated in this population or in other pediatric groups with unique neurocognitive and neurobehavioral symptoms. And the same sensitivity issue arises with adaptive measures as with neurocognitive measures in that the currently available measures may not be sensitive enough to detect incremental change. Moreover, standard measures also can be unreliable in pediatric patient populations due to poor patient cooperation, parental/caregiver bias, and the failure most trials to control for variables such as the potential medical trauma to children who are already subjected to regular medical interventions. Finally, the challenges of accurately evaluating efficacy of experimental therapies is not limited to neurodegenerative disease that affect children. Studies in other neurodegenerative diseases, such as Alzheimer's disease and Parkinson's disease, also suffer from sensitivity issues in attempts to capture neurocognitive improvements, stability, or decline when compared to controls.

There is little research on the precise relationship between functional ability and cognition in children. Toileting implicates cognitive ability (the ability to recognize a task needs to be done, planning to do it, and carrying it out), physical ability (ability to complete the tasks), perception (sensory input regarding the necessity of a task and its completion), and behavioral control (ability to align one's actions with one's cognitive understanding, intentions, and physical ability).

In at least one aspect, methods of evaluating neurocognitive function in at least one patient suspected of having or diagnosed with a neurodegenerative disease are described herein. The methods include collecting data that indicates an ability of the at least one patient to complete a plurality of toileting tasks; and using the data to generate a report that describes a neurocognitive function in the at least one patient, thereby using the toileting tasks as a nontraditional measures of neurocognitive function. The methods have utility for evaluating the effectiveness of clinical therapies, and the methods optionally further include administering a therapy to the one or more patients. The methods also have utility for evaluating changes in neurocognitive function, including the progression of a neurocognitive disease in a patient or patient population. Accordingly, in some methods disclosed herein, a step of collecting data can include collecting a first data set, a second data set, and optionally a third data set, where the first, second, and optional third data sets are collected at different times.

Neurodegenerative diseases include, but are not limited to lysosomal storage diseases, Alzheimer's disease, Parkinson's disease, Huntington's disease, prion disease, and Lewy body disease. MPS II, discussed in detail herein, and other mucopolysaccharidoses are lysosomal storage diseases. These diseases can involve neurological complications, including impaired motor function, which makes common daily tasks difficult or impossible for the affected patient. As disclosed herein, tasks related to toileting skills directly correlates to neurocognitive function. That is, greater skill in accomplishing toileting tasks indicates better neurocognitive function, and declining abilities to independently complete toileting tasks indicates declining neurocognitive function. The disclosed methods use toileting abilities, as nontraditional surrogate measures of neurocognitive functions. Remarkably, these nontraditional measures provide greater accuracy and precision in evaluating neurocognitive function and assessing neurodegenerative disease progression than traditional neurocognitive and adaptive measures.

Tasks related to toileting skills include understands potty words, stays clean (no bowel movement accidents) while asleep, shows interest in using the toilet or potty chair, flushes the toilet by himself, indicates during or after having a bowel movement, stays dry for over 2 hours, indicates the need to go to the bathroom, knows how to urinate in the toilet, sits on toilet when placed for at least 1 minute, urinates in toilet with help, indicates during or after urinating, pulls training pants or underwear down by self, pulls training pants or underwear up by self, indicates before having a bowel movement, wears training pants or underwear, uses regular toilet without a child seat, stays bowel movement accident free during day, indicates before having to urinate, stays dry during the day, wakes up dry overnight, enters bathroom and urinates by self, urinates while standing by self (boy) or wipes urine effectively by self (girl), enters bathroom and has bowel movement by self, initiates wiping after using the toilet, wipes poop effectively by self, initiates washing hands after using the toilet, washes hands independently after using the toilet.

The precise language used to describe some of the toileting skills listed above can be important for certain patient populations. This is particularly true where that language is used in a survey where a patent or caregiver is assigning scores to represent the patient's ability to do various toileting tasks, and the scores will be used in evaluating the patient's neurocognitive function. For example, if a patient lacks verbal skills but is able to effectively communicate in some other way, and a task is described as “say that s/he needs to go to the bathroom,” the patient or caregiver may score that task low based on lack of verbal skill instead of scoring based on the toileting skill, which would introduce error and uncertainty in the evaluation. Thus, describing the task as “indicate the need to go to the bathroom” would lead to a more accurate and precise evaluation. Similarly, tasks described in simple language, or child-like language, are more likely to be understood and accurately scored where a patient who may be somewhat cognitively impaired is assigning scores for his own abilities. Thus, in some examples, a task may be described using the word potty instead of the word toilet and/or using the word pee instead of urinate. Finally, because it is important that the measures be sensitive enough to indicate incremental changes in neurocognitive abilities, it is also important to separately assess abilities to complete tasks that differ only incrementally in difficulty. Thus, while an assessment of toileting abilities of a neurotypical individual might include scoring the individual's ability to wash his/her hands after using the toilet, to evaluate neurocognitive function in a cognitively impaired individual, that task should be described more precisely and ideally should be broken up into multiple tasks that differ in difficulty, such as “initiates washing hands after using the toilet” and “washes hands independently after using the toilet.”

In some aspects, the methods disclosed herein collect data that indicates an ability of a patient or patient population to complete at least two, at least three, at least four, at least five, or at least six, or all of the toileting tasks selected from (a) initiate washing hands after using the toilet, (b) initiate wiping after using the toilet, (c) indicate during or after having a bowel movement, (d) indicate before having to urinate, (e) indicate before having a bowel movement, (d) indicate during or after urinating, and (e) sit on the toilet when placed for at least 1 minute. Optionally, the data indicates the ability of the patient or patent population to complete both of (a) initiate washing hands after using the toilet and (f) wash hands independently after using the toilet or both of (b) initiate wiping after using the toilet and (g) wipe poop effectively by self.

A step of collecting data that indicates an ability of the at least one patient to complete a plurality of toileting tasks in any method described herein can involve, but does not require, assessment of the patient by the individual or entity collecting the data. For example, the step of collecting data may include observing a patient while the patient completes a plurality of toileting tasks and assigning a score for each toileting task, where the score indicates the ability of the patient to complete the task. As other examples, the step of collecting data may include one or more of drafting a plurality of questions that seek an indication of a patient's ability to complete each of a plurality of toileting tasks (i.e., a “toileting survey”), distributing one or more blank toileting surveys, receiving one or more completed toileting surveys. In other examples, a step of collecting data can be accomplished by receiving the data. The data may be received from any individual or entity, such as an individual who completes a toileting survey, and including but not limited to the patient, a caregiver, a healthcare provider. Alternatively, the data may be received from any individual or entity that received the data from the patient, a caregiver, or a healthcare provider. For example, the step of collecting data can be accomplished by receiving the data from an individual or entity that receives, compiles, and/or stores data related to a plurality of patients.

The data is not limited to any specific form of data. In any method described herein, the step of collecting data can be accomplished by receiving data in oral, written, electronic, or any other useful form. As one example, the data can be provided by a patient or caregiver in response to a survey, where answers are provided orally, in writing, or electronically, such as through a web-based platform.

Optionally, a step of collecting data can be accomplished by receiving data in the form of individual numerical scores for each task and for each patient. Alternatively, those individual scores can be considered raw data, and the step of collecting data can be accomplished by receiving adjusted data, such as raw data that has been averaged, or raw data that is presented in the form of a percentage. Data is adjusted data when it is derived from but not identical to the raw data, such as when at least some of the raw data scores are averaged. Raw data may be adjusted for any reason, such as to ensure the raw data scores cannot be connected with a specific individual or to facilitate analysis by the entity collecting the data. Adjusted data may include fewer data points as compared to the corresponding raw data, such as when at least some scores are averaged. Adjusted data may include more data points as compared to the corresponding raw data, such as when confidence intervals or statistical measures are added to data points.

In some aspects, data indicates an ability of a patient to complete a toileting task when the patient's ability to complete the task is given a numerical score on a defined or known scale. The numerical scores can be based on any desired or convenient scale that can represent ability. Optionally, the scores are based on a Likert scale, where the score indicates the frequency with which the patient can perform the task. For example, if the patient never performs the task, the ability would be given the lowest score, generally a 1. If the patient can always perform the task, the ability would be given the highest score. For a Likert scale, the highest score is an odd number, such as 3, 5 or 7. The methods described herein can be used with any convenient scale for rating patient ability. In some aspects, the methods are used with a Likert scale of 1 to 3, 1 to 5, or 1 to 7.

After the data is collected, can be used to evaluate neurocognitive function. The step of using the data to evaluate neurocognitive function in a patient can include any comparison and/or mathematical manipulation of the collected data that can indicate a status of or change in the neurocognitive function of at least one patient. As one example, using the data to evaluate neurocognitive function can include calculating a Toileting Abilities Percentage (“TAP”). TAP is a representation of one or more raw data scores as a percentage of the highest possible score. TAP can be calculated for a single patient, for a subset of a patient population, and/or as an overall TAP for an entire patient population. TAP can relate to a single task, a subset of all tasks assessed, or all tasks assessed. TAP can be used as a primary outcome and as a continuous measure for evaluating disease progression and effectiveness of clinical therapies.

In one nonlimiting example, 100 patients are evaluated on their abilities to perform 30 individual tasks. Each patient is scored on a five-point Likert scale for each task. The highest possible score for any patient is 30 tasks×5 points=150=100%. The lowest possible score for any patient is 30 tasks×1 point=30=0%. A patient with a total score of 105 points would have a TAP of (105−30)/(150−30)=62.5%, and a patient with a total score of 45 points would have a TAP of (45−30)/(150−30)=12.5%.

Another example of using the data to evaluate neurocognitive function is comparing the data to analogous data collected at a different time for the same patient or patient population. In this case, the methods disclosed herein can evaluate the progression of a neurodegenerative disease in a single patient or in a patient population. A further example of using the data to evaluate neurocognitive function comparing data collected from a patient population receiving a certain therapy to analogous data collected from a patient population not receiving the therapy. In this case, the method can be used to evaluate the effectiveness of the therapy.

Any evaluation of neurocognitive function described herein can be described or the results documented in a report. Thus, in at least one aspect, the invention provides methods for generating a report for diagnosing a neurodegenerative disease or condition or evaluating a progression of the neurodegenerative disease or condition associated with reduced neurocognitive function in a patient. One example of such a disease or condition is MPS II. Other examples of such a disease or condition include other lysosomal storage diseases, Alzheimer's disease, Parkinson's disease, Huntington's disease, prion disease, or Lewy body disease. Such a method may include the steps of collecting data that indicates an ability of the at least one patient to complete a plurality of toileting tasks; and using the data to generate a report that describes a neurocognitive function in the at least one patient, thereby using the plurality of the toileting tasks as nontraditional surrogate measures of the neurocognitive function in the at least one patient. The methods may further comprise generating a report that recites the ability of the patient to complete a plurality of toileting tasks. Based on the report on the ability of the patient to complete a plurality of toileting tasks, one could assess whether a subject has an abnormally low neurocognitive function, or whether the subjects neurocognitive function is incrementally decreasing, staying constant, or increasing. Such information can be useful for diagnosing a neurodegenerative disease or condition, evaluating a progression of the neurodegenerative disease or condition associated with reduced neurocognitive function in the patient, or evaluating a therapy for treating the neurodegenerative disease or condition.

Accordingly, in some aspects, described herein are methods of evaluating neurocognitive function in one or more patients suspected of having or diagnosed with a neurodegenerative disease, where the methods include collecting a first data set at a first time and collecting a second data set at a second time, wherein the first data set and the second data set indicate abilities of the one or more patients to complete a plurality of toileting tasks, and wherein the second time is at least two months after the first time; and using the first data set and the second data set to evaluate the progression of the neurodegenerative disease in the one or more patients, thereby using the toileting tasks as nontraditional surrogate measures of neurocognitive function.

In some examples, the first and second data sets are collected about three months, about six months, about nine months, or about one year apart. Thus, in some examples, the second time is about three months, about six months, about nine months, or about one year after the first time. The method can further include collecting a third data set at a third time, where the third time is at least two months after the second time. Optionally the third time is about three months, about six months, about nine months, or about one year after the second time.

When data collected from a patient population receiving a certain therapy is compared to analogous data collected from a patient population not receiving the therapy, the method can be used to evaluate the effectiveness of the therapy.

Accordingly, in some aspects, described herein are methods of evaluating the effectiveness of a therapy for a neurodegenerative disease, where the methods include administering a therapy to a patient population that includes patients suspected of having or diagnosed with the neurodegenerative disease; collecting a first data set that indicates abilities of the treated patient population to complete a plurality of toileting tasks; and comparing the first data set to a second data set, wherein the second data set indicates abilities of a control untreated patient population to complete the plurality of toileting tasks, wherein the control untreated patient population includes patients suspected of having or diagnosed with the neurodegenerative disease, thereby evaluating the effectiveness of the therapy and using the toileting tasks as a nontraditional measure of neurocognitive function.

In some examples, the first and second data sets are collected about three months, about six months, about nine months, or about one year apart. Thus, in some examples, the second time is about three months, about six months, about nine months, or about one year after the first time. The method can further include collecting a third data set at a third time, where the third time is at least two months after the second time. Optionally the third time is about three months, about six months, about nine months, or about one year after the second time.

Aspects of the invention provide assessment tools for evaluating the cognitive status of an individual patient or a defined patient group.

As noted herein, in certain embodiments, the invention may comprise methods of developing and using a survey of nontraditional surrogate measures of cognitive function. The methods of the invention may be embodied in a variety of ways.

### EXAMPLES

The following examples demonstrate embodiments of development and application of a surrogate measures survey for evaluating neurocognitive function.

### Example 1. Development of Surrogate Measures and Survey

Traditional neurocognitive measures do not necessarily reflect meaningful improvement to patients and caregivers for certain populations. The USFDA has requested that clinical trials measure outcomes that have relevance to the real-world experience of patients and families. To initiate development of such a measure, caregivers of patients with MPS II were interviewed, including caregivers of patients who were enrolled in studies of intrathecal idursulfase and those who were receiving standard therapy (i.e., intravenous enzyme replacement therapy with idursulfase).

Caregivers identified toileting as a skill that was important to them due to the time, effort, and cost associated with caring for a child without the ability to independently toilet. Caregivers discussed the time spent attempting to toilet train children before an MPS II diagnosis, extended time spent (compared with typical children) attempting toilet training after an MPS II diagnosis (if attempted at all), and their child's lack of cognitive understanding of the sensation of wet and soiled diapers or the steps required to toilet. Caregivers also discussed the time and effort required to identify suitable continence products, diaper progressively larger children, train support staff, address accidents, and the challenge of managing incontinence at school and with travel. The cost of diapers, wipes, extra diaper liners, and bed liners was also of concern, considering few of these products are covered by private insurance (although some are covered by Medicaid).

Discussions in multiple social media groups for MPS II individuals and families were also reviewed. Specifically, they observed and described differences in conversations from groups that consisted solely of caregivers of patients receiving an experimental therapy, intrathecal idursulfase, and those groups where the majority of members were not involved in the use of that experimental therapy. Those conversations differed on many topics, but distinctly, with respect to the task of toileting and toilet training. In the groups where children were receiving intrathecal idursulfase, the discussions focused predominantly on toilet training, fully independent toileting, the use of “pull-ups,” training regimens in school, and to a lesser extent, continence products. Such conversations differed in tone and substance from those occurring in the groups of caregivers of patients not on intrathecal idursulfase, which focused predominantly on diapers and continence products with some discussion related to the possibility of toilet training in younger ages or trip-training, and also the futility of attempting toilet training in neuronopathic patients.

The Toileting Abilities Survey (TAS) considered and included study aspects from the Fundamentals of Toileting Training Study (FTTS), a longitudinal study of 267 typically-developing children published in the journal Pediatrics. The FTTS used an instrument called the Training Status Survey (TSS) that parents completed on a weekly basis for 12 to 16 months (median of 49 per child). The TSS was “developed, piloted, revised, and used” for the FTTS project, which consisted of 26 skills common to both genders and 1 skill specific to each gender (“wipes urine effectively” for girls and “urinates while standing” for boys), rated on a 5-point Likert scale (1=never, 2, 3=sometimes, 4, 5=always). The study resulted in an ordered progressive acquisition of the skills toward fully independent toileting, for both boys and girls.

The wider array of 27 skills noted in the overall acquisition of independent toileting was much greater than any measure reviewed (note the MPS-HAQ included four questions, and the VABS-III includes eight questions related to toileting). Therefore, the potential for much greater sensitivity than current measures was evident. In addition, the five-point Likert scale offered additional sensitivity over the three-point Likert scale used in the VABS measures or similar surveys.

The Training Status Survey was initially launched as part of an online survey initiated by Project Alive, a nonprofit MPS II research and advocacy foundation. The survey was completed by 121 primary caregivers of patients with MPS II, including 42 caregivers of patients who had received intrathecal idursulfase.

A focus group was then conducted to evaluate the individual questions and skills assessed, resulting in the removal of two questions, the addition of two new questions, and the revision of 16 questions to make them more appropriate for neurodegenerative diseases such as MPS II, and to MPS II particularly in some cases. This resulted in the toileting skills questions of the Toileting Abilities Survey.

The TAS Survey includes the following text:

### Example 2. Application of Surrogate Measures to Evaluation of a Therapy for MPS II Patients

The developed survey of surrogate measures, TAS, was applied to evaluate whether a new caregiver outcome measure assessing toileting abilities is a valid method to capture change associated with the administration of an experimental therapy, intrathecal idursulfase, in the neuronopathic MPS II population.

The Toileting Abilities Survey (TAS), which in addition to toileting skills, covers demographic data, diagnosis, disease form and symptoms, and the use of assistive devices and treatments (including standard of care and experimental therapies), was administered to caregivers of individuals with MPS II over a period of two weeks in August 2019. Data collection for this study was conducted on the Backpack Health application and was designed to be administered to caregivers approximately every three months from the last date of completion. Described here is the data collection from timepoint one only.

Backpack Health, a division of Konica Minolta Precision Medicine, offers mobile and web-based software that can be used by individuals or those acting on their behalf (e.g., parents, non-healthcare provider caregivers) to store and update health information (e.g., conditions and symptoms, medications, treatments and therapies, procedures and surgeries, allergies, vaccinations, measurements and laboratory and test results) and to create health profile entries.

Backpack Health also offers users the ability to complete surveys from within the app. Once users have completed a survey for themselves or someone for whom they care, they are given the option to add relevant health information collected during the survey to their Backpack Health profile or their care recipient's profile. This functionality allows users to manage and store all of their and their care recipients' health information in one central location.

While Backpack Health users were asked to enter full or partial dates when completing the TAS, this information was not provided directly to the authors for this study. Instead, only years, or dates shifted using the offset date method were provided. Because of this study design, ages and time periods were calculated with 2019 being the current date (data being collected in Q3 2019) and by subtracting and averaging years.

Backpack Health users who join themselves or their loved one living with MPS II to the Hunter Syndrome International Patient Registry were notified of the availability and location of the TAS via secure in-app messaging and email notifications. No Backpack Health user contact information (e.g., email address) and no personally identifiable information of any Backpack Health users or TAS respondents was shared with researchers.

The study assessed the Toileting Abilities Percentage (TAP), calculated as the percentage of the highest score possible across the entire survey (27 skills×5-points on the Likert scale, reflecting “always” does all skills independently=135) as 100%, with the minimum (27 skills×1-point on the Likert scale, reflecting “never” does any of the skills independently=27) as 0%. The study also assessed the mean on each of the individual 27 toileting abilities. Comparisons were then performed of the groups treated with both idursulfase and intrathecal idursulfase (Treatment Group) and those treated with idursulfase only (Control Group).

For statistical analysis, Toileting Abilities Percentage was used as the primary outcome and as a continuous measure. The 5-point Likert scale for the 27 individual toileting abilities were assessed both as interval scale measures and as categorical outcomes. Descriptive statistics were computed for each of the Toileting Abilities Percentage (TAP) and the individual 27 toileting abilities. Descriptive statistics included mean, standard deviation, min, 25th percentile, median, 75th percentile, max, 95% CI, and frequency/proportions. Pearson and Spearman rank correlations were computed between the outcomes (individual 27 toileting abilities, TAP). Two-sample t-tests were used to compare outcomes between those on intrathecal idursulfase (N=32) and those not on intrathecal idursulfase (N=54). Multiple linear regression models were used as model outcomes using joint predictors. TAP and the 27 individual toileting abilities were used as dependent variables and predictors were treatment (intrathecal idursulfase or not), age, time on idursulfase, time since diagnosed with MPS II, interaction between treatment and age, and interaction between treatment and time on idursulfase.

### Neuronopathic Study Population

Of the 186 responses to the TAS initially received, 59% (n=110) described their child as having been diagnosed with the severe, neuronopathic, or cognitively-regressive form of MPS II, 24% (n=45) described their child as having attenuated or non-neuronopathic form, and 17% (n=31) reported they didn't know which form their child had.

In the caregiver-identified neuronopathic population, the most common cognitive and behavioral symptoms experienced in the past month were delayed speech, cognitive delay, short attention span, repetitive behaviors, and reduced gross motor planning.

In the caregiver-identified non-neuronopathic population, cognitive and behavioral symptoms were still present, with short attention span in almost half of the population, and hyperactivity, delayed speech, cognitive delay, and repetitive behaviors in approximately one-third of the non-neuronopathic population.

Regarding treatments, 91% of the overall population had received idursulfase as a therapy with 93% of those continuing on the therapy as of the survey date. With respect to other treatments, 19% reported having received intrathecal idursulfase, 5% reported having received HSCT or bone marrow transplant, 1% reported having received idursulfase beta, and 1% reported having received JR-141.

A number of patients were eliminated from the study, including 76 respondents who were reported by their primary caregivers as being attenuated or unsure of their form of disease as well as 24 additional patients for other reasons. This process resulted in 86 relevant patients in the neuronopathic population, 32 of whom were reported by their caregivers as currently receiving both idursulfase and intrathecal idursulfase (treatment), and 54 of whom were reported by their caregivers as receiving idursulfase (control).

The population was evaluated for the range and mean of current age, age at start of idursulfase, and time on idursulfase, as shown in Table 1.

TABLE 1
Population Characteristics

| | | | Mean | Age | | |
|---|---|---|---|---|---|---|
| | | | age at | range at | Mean | Range of |
| | Mean | Current | idursulfase | idursulfase | time on | time on |
| Group | current age | age range | Start | Start | idursulfase | idursulfase |
| Treatment | 9.3 years | 4-19 years | 2.7 years | 0-7 years | 6.7 years | 3-13 years |
| Control | 9.8 years | 2-22 years | 3.6 years | 1-11 years | 6.2 years | 1-13 years |

The treatment population was also evaluated for age at intrathecal idursulfase start and time on intrathecal idursulfase, as shown in Table 2.

TABLE 2
Additional Characteristics of Treatment Group

| | Mean age at | Age range at | Mean | Range of |
|---|---|---|---|---|
| | intrathecal | intrathecal | time on | time on |
| | idursulfase | idursulfase | intrathecal | intrathecal |
| Group | start | start | idursulfase | idursulfase |
| Treatment | 4.8 years | 1-11 years | 4.5 years | 3-9 years |

### Toileting Abilities Percentage (TAP)

The analysis of the overall Toileting Abilities Percentage or TAP of neuronopathic patients receiving intrathecal idursulfase (n=32) reflect a mean TAP of 61% independent toileting, which, compared with neuronopathic patients receiving only idursulfase (n=54), reporting a mean TAP of 22% independent toileting, is statistically significant with a t-test of <0.0001.

A linear regression model to control for age and time on idursulfase shows that neither are statistically significant predictors and the test for treatment difference is P-value<0.0001. Therefore, the difference in overall toileting abilities of those neuronopathic MPS II patients receiving idursulfase and intrathecal idursulfase versus those receiving only idursulfase is statistically significant. There was no statistically significant association between age or time on intrathecal idursulfase and the TAP.

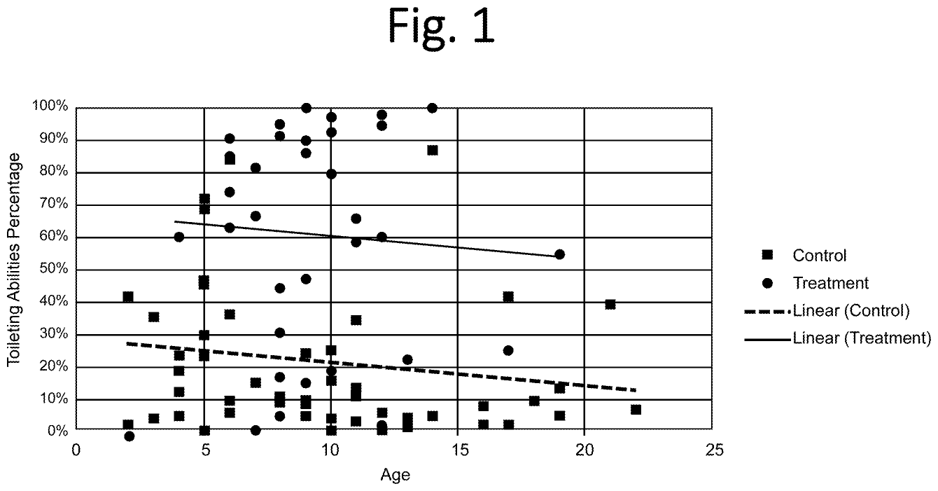

FIG. 1 illustrates that there were significantly greater TAP scores for the treatment population, specifically that 47% ( 15/32) of the treatment group, versus 4% ( 2/52) of the control group, had TAP scores greater than 75%. Lowering the threshold, 62% ( 20/32) of the treatment group, versus 13% ( 7/52) of the control group, had TAP scores greater than 50%. This represents significantly greater toileting abilities in the treated patients.

### Individual Toileting Abilities

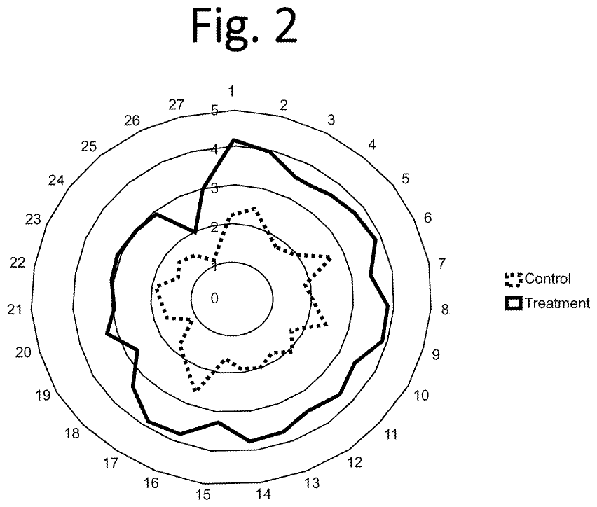

FIG. 2 illustrates, within each of the 27 individual toileting skills measured in the TAS, the mean of treated patients (n=32) exceeding that of untreated patients (n=54), rising to statistical significance in every individual skill. That statistical significance is also maintained even when controlling for age and time on idursulfase.

### Patients Beginning Treatment at 6 Years of Age or Older

Given the above data, and the broad age ranges of the population, the goal was to identify whether the improvement in toileting abilities holds true for patients who initiated intrathecal idursulfase at an older age, after which cognitive abilities were expected to have begun their decline. A review of the literature suggests that although significant heterogeneity can exist in the cognitive and behavioral symptoms, and there is often a plateau of these symptoms generally expected to begin between the ages of 3-5 years old, with decline generally expected to begin thereafter. Therefore, 6 years of age was selected as an appropriate starting point for older patients who, on average, would not be expected to be maintaining or gaining cognitive or adaptive skills in neuronopathic MPS II.

A total of 7 patients in the treated population initiated intrathecal idursulfase treatment at age 6 years or older (“Older Treatment Group”) and their characteristics are detailed in Table 3.

TABLE 3
Subject Characteristics of Older Treatment Group -
Individual Patients A-G (data in years)

| | Mean | Std Dev | A | B | C | D | E | F | G |
|---|---|---|---|---|---|---|---|---|---|
| Current Age | 12.29 | 4.19 | 9 | 9 | 9 | 10 | 13 | 17 | 19 |
| Age at idursulfase start | 3.71 | 1.98 | 5 | 4 | 1 | 2 | 3 | 4 | 7 |
| Time on idursulfase | 8.57 | 3.36 | 4 | 5 | 8 | 8 | 10 | 13 | 12 |
| Age at intrathecal | 7.86 | 2.12 | 6 | 6 | 6 | 7 | 9 | 10 | 11 |
| idursulfase start | | | | | | | | | |
| Time on intrathecal | 4.43 | 2.15 | 3 | 3 | 3 | 3 | 4 | 7 | 8 |
| idursulfase | | | | | | | | | |

A comparator group within the control group (an “Older Control Group”) was identified in order to analyze how initiating intrathecal idursulfase at 6 years of age or older might impact toileting abilities. The goal was a comparator population at 2-1 of the treated population, with similar current age, age at idursulfase start, and time on idursulfase. Where identical values were not possible, conservative estimates in favor of the control population (control group having lower current age, lower age at idursulfase start, and longer time on idursulfase) were employed.

To create the comparator group, all patients in the overall control group that were age-matched to patients in the Older Treatment Group were first identified. Each of the treated patients in the Older Treatment Group had exactly 2:1 current age-matched comparators, with the exception of patient B, with no current age-matched comparators, and patient D, which had five current age-matched comparators. For patient B, (the only) two control patients were identified who were one year younger in current age, with the remaining values being more conservative values, thereby potentially benefiting the control as described above. For patient D, of the five age-matched comparators, the two patients with the closest values in age at idursulfase start and time on idursulfase were selected. Table 4 reflects the characteristics of the Older Treatment and Older Control groups.

TABLE 4
Means (years) of Older Treatment
Group versus Older Control Group

| | Older | Older | |
|---|---|---|---|
| | Treatment | Control | |
| | Group (n = 7) | Group (n = 14) | Std Dev |
| Current Age | 12.29 | 12.14 | 4.17 |
| Age at idursulfase start | 3.71 | 3.71 | 1.86 |
| Time on idursulfase | 8.57 | 8.50 | 3.34 |
| | | | (mean 8.43) |
| Age at intrathecal | 7.86 | | |
| idursulfase start | | | |
| Time on intrathecal | 4.43 | | |
| idursulfase | | | |

Given the Older Control Group's population reflecting slightly younger mean current age, the same mean age at idursulfase start, and very similar time on idursulfase, one would expect that absent a treatment effect, the groups would have similar toileting abilities.

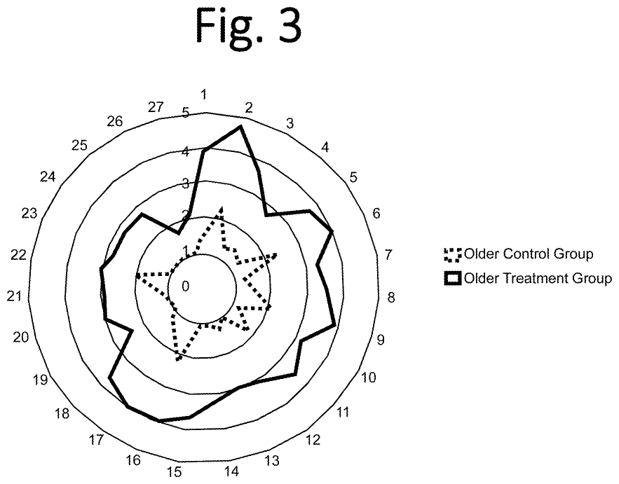

However, as FIG. 3 shows, the Older Treatment Group exhibited significantly higher and statistically significantly greater Toileting Abilities Percentage, 56% for the Older Treatment Group versus 10% for the Older Control Group, with a p value of 0.0002. Similar to the overall treatment group, the Older Treatment Group exhibited higher individual toileting skills, with every skill reaching statistical significance.

Utilizing the TAS to measure treatment benefit of intrathecal idursulfase exhibited sensitivity within individual toileting skills, as well as overall, represented by the Toileting Abilities Percentage (TAP). Treatment with idursulfase and intrathecal idursulfase is associated with significantly higher individual and overall toileting skills versus treatment with idursulfase alone.

The significant difference in TAP between the treatment and control groups (61% versus 22%) suggests that intrathecal idursulfase positively impacts the functions (cognitive and otherwise) necessary for toileting. That difference is represented across the entire span of individual toileting abilities, with similar patterns of higher and lower abilities represented in both the treatment and control groups. These patterns suggest that the measure detects individual skills that are easier or more difficult to attain across the board and support the sensitivity of the measure. While the control group was slightly older, started idursulfase at a slightly older age, and were on idursulfase for slightly less time, none of those factors varied the statistical significance of intrathecal idursulfase as the controlling factor in the increased toileting abilities.

Secondarily, in examining those patients who began receiving intrathecal idursulfase at age six or older, the goal was to determine whether starting the therapy at an older age would impact the benefit to toileting skills. While the TAP was slightly less in the older population in comparison to the control group, each individual toileting skill, as well as the overall Toileting Ability Percentage, remained statistically significant, and the overall treatment difference of the TAP was actually even larger.

In the overall neuronopathic population, the treatment group fared 39 percentage points higher than the control group. By removing the Older Population (Older Treatment Group and Older Control Group), in the remaining patients, termed the Younger Population, the differential between the treatment and control groups dropped to 37 points, whereas the differential in the Older Population was 46 points. So while the patients who started intrathecal idursulfase at an older age (age 6 years and over) did not exhibit a TAP as high as those who started treatment at a younger age, the difference when compared to the untreated control population is greater. Therefore, there is the potential for significant benefit even to older patients in beginning treatment with intrathecal idursulfase.

TABLE 5
TAP scores for Overall and Age-Parsed Populations

| | Treatment | Control |
|---|---|---|
| Overall Population | 61% (n = 32) | 22% (n = 54) |
| Older Population | 56% (n = 7) | 10% (n = 14) |
| Younger Population | 62% (n = 25) | 25% (n = 40) |

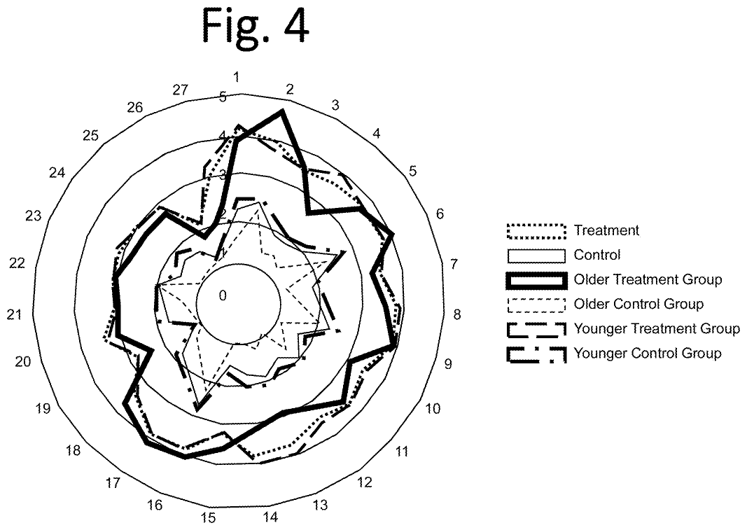

In addition, the Older Treatment Group still has significantly greater individual toileting skills than even the younger control group, or the current-age matched control group. FIG. 4 shows mean scores on the TAS (5-point Likert scale) for each of the 27 Individual toileting abilities by treatment status for overall and age-parsed populations.

Nonlimiting embodiments include:

1. A method of evaluating neurocognitive function in at least one patient suspected of having or diagnosed with a neurodegenerative disease comprising, collecting data that indicates an ability of the at least one patient to complete a plurality of toileting tasks; and using the data to generate a report that describes a neurocognitive function in the at least one patient, thereby using the plurality of the toileting tasks as nontraditional surrogate measures of the neurocognitive function in the at least one patient.

2. The method of embodiment 1, wherein the step of collecting the data comprises collecting a first data set at a first time, and collecting a second data set at a second time, wherein the second data set indicates an ability of the at least one patient to complete the plurality of the toileting tasks at the second time, wherein the second time is at least two months after the first time, and wherein the step of using the data to generate a report comprises indicating a progression of the neurodegenerative disease in the at least one patient based on a comparison of the first data set and the second data set.

3. The method of any preceding embodiment, wherein the at least one patient comprises a first patient population, wherein the step of collecting the data comprises collecting a first data set, and wherein the method further comprises before collecting the first data set, administering a therapy to the first patient population, wherein the step of using the data to generate a report comprises indicating effectiveness of the therapy based on comparing the first data set to a second data for a control patient population, wherein the second data set indicates an ability of the control patient population to complete the plurality of the toileting tasks, wherein the control patient population includes patients suspected of having or diagnosed with the neurodegenerative disease, and wherein the control patient population has not received the therapy.

4. The method of any preceding embodiment, wherein the neurodegenerative disease comprises a lysosomal storage disease, Alzheimer's disease, Parkinson's disease, Huntington's disease, prion disease, or Lewy body disease.

5. The method of any preceding embodiment, wherein the neurodegenerative disease comprises a lysosomal storage disease comprising a mucopolysaccharidosis, in particular mucopolysaccharidosis type II (MPS II).

6. The method of any preceding embodiment, wherein the plurality of the toileting tasks comprises at least two, at least three, at least four, at least five, at least six, or all of: initiate washing hands after using a toilet, initiate wiping after using the toilet, indicate during or after having a bowel movement, indicate before having to urinate, indicate before having a bowel movement, indicate during or after urinating, sit on the toilet when placed for at least 1 minute.

7. The method of any preceding embodiment, wherein the plurality of the toileting tasks comprises both of (a) initiate washing hands after using the toilet and (f) wash hands independently after using the toilet, or both of (b) initiate wiping after using the toilet and (g) wipe poop effectively by self.

8. The method of any preceding embodiment, wherein the step of collecting the data comprises observing a patient while the patient completes a plurality of toileting tasks and assigning a score for each toileting task, where the score indicates the ability of the patient to complete the task.

9. The method of any preceding embodiment, wherein the step of collecting the data comprises receiving the data from the at least one patient, a caregiver, or a healthcare provider; or receiving the data from an individual or entity that received the data from the at least one patient, the caregiver, or the healthcare provider; or receiving the data from an individual or entity that receives, compiles, and/or stores health information related to a plurality of patients.

10. The method of any preceding embodiment, wherein the step of collecting the data comprises receiving an individual numerical score for each individual patient and for each toileting task of the plurality of the toileting tasks or receiving data derived from one or more of the individual numerical scores, wherein each individual numerical score indicates an ability of the individual patient to complete the toileting task.

11. The method of any preceding embodiment, wherein the individual numerical scores comprise scores of from 1 to 3, 1 to 5, or 1 to 7 on a Likert scale.

12. The method of any preceding embodiment, wherein the step of using the data to generate a report comprises indicating a Toileting Abilities Percentage (“TAP”) to represent one or more of the individual numerical scores as a percentage of a highest possible score.

13. The method of any preceding embodiment, further comprising administering a therapy to the at least one patient after collecting the first data set at the first time and before collecting the second data set at the second time.

14. The method of any preceding embodiment, wherein the second time is about three months, about six months, about nine months, or about a year after the first time.

15. The method of any preceding embodiment, wherein the step of collecting the data further comprises collecting a third data set from the at least one patient at a third time, wherein the third data set indicates an ability of the at least one patient to complete a plurality of toileting tasks at the third time, wherein the third time is at least two months after the second time, and wherein the step of using the data to generate a report that describes the neurocognitive function in the at least one patient comprises indicating the progression of the neurodegenerative disease in the at least one patient based on a comparison of the first data set, the second data set, and the third data set.

16. The method of any preceding embodiment, wherein the second time is about three months, about six months, about nine months, or about a year after the first time, wherein the third time is about three months, about six months, about nine months, or about a year after the second time.

17. The method of any preceding embodiment wherein the therapy comprises enzyme replacement, gene therapy, gene editing, a fusion protein, substrate reduction therapy, or a chaperone molecule.

1. A method of evaluating neurocognitive function in at least one patient suspected of having or diagnosed with a neurodegenerative disease comprising,
collecting data that indicates an ability of the at least one patient to complete a plurality of toileting tasks; and
using the data to generate a report that describes a neurocognitive function in the at least one patient,
thereby using the plurality of the toileting tasks as nontraditional surrogate measures of the neurocognitive function in the at least one patient.

2. The method of claim 1, wherein the step of collecting the data
comprises collecting a first data set at a first time, and
collecting a second data set at a second time,
wherein the second data set indicates an ability of the at least one patient to complete the plurality of the toileting tasks at the second time,
wherein the second time is at least two months after the first time, and
wherein the step of using the data to generate a report comprises indicating a progression of the neurodegenerative disease in the at least one patient based on a comparison of the first data set and the second data set.

3. The method of claim 1, wherein the at least one patient comprises a first patient population, wherein the step of collecting the data comprises collecting a first data set, and wherein the method further comprises
before collecting the first data set, administering a therapy to the first patient population,
wherein the step of using the data to generate a report comprises indicating effectiveness of the therapy based on comparing the first data set to a second data for a control patient population,
wherein the second data set indicates an ability of the control patient population to complete the plurality of the toileting tasks,
wherein the control patient population includes patients suspected of having or diagnosed with the neurodegenerative disease, and
wherein the control patient population has not received the therapy.

4. The method of claim 1, wherein the neurodegenerative disease comprises a lysosomal storage disease, Alzheimer's disease, Parkinson's disease, Huntington's disease, prion disease, or Lewy body disease.

5. The method of claim 4, wherein the neurodegenerative disease comprises a lysosomal storage disease comprising a mucopolysaccharidosis, in particular mucopolysaccharidosis type II (MPS II).

6. The method of claim 1, wherein the plurality of the toileting tasks comprises at least two, at least three, at least four, at least five, at least six, or all of: initiate washing hands after using a toilet, initiate wiping after using the toilet, indicate during or after having a bowel movement, indicate before having to urinate, indicate before having a bowel movement, indicate during or after urinating, sit on the toilet when placed for at least 1 minute.

7. The method of claim 1, wherein the plurality of the toileting tasks comprises both of (a) initiate washing hands after using the toilet and (f) wash hands independently after using the toilet, or both of (b) initiate wiping after using the toilet and (g) wipe poop effectively by self.

8. The method of claim 1, wherein the step of collecting the data comprises observing a patient while the patient completes a plurality of toileting tasks and assigning a score for each toileting task, where the score indicates the ability of the patient to complete the task.

9. The method of claim 1, wherein the step of collecting the data comprises receiving the data from the at least one patient, a caregiver, or a healthcare provider; or receiving the data from an individual or entity that received the data from the at least one patient, the caregiver, or the healthcare provider; or receiving the data from an individual or entity that receives, compiles, and/or stores health information related to a plurality of patients.

10. The method of claim 1, wherein the step of collecting the data comprises receiving an individual numerical score for each individual patient and for each toileting task of the plurality of the toileting tasks or receiving data derived from one or more of the individual numerical scores, wherein each individual numerical score indicates an ability of the individual patient to complete the toileting task.

11. The method of claim 10, wherein the individual numerical scores comprise scores of from 1 to 3, 1 to 5, or 1 to 7 on a Likert scale.

12. The method of claim 10, wherein the step of using the data to generate a report comprises indicating a Toileting Abilities Percentage (“TAP”) to represent one or more of the individual numerical scores as a percentage of a highest possible score.

13. The method of claim 2, further comprising administering a therapy to the at least one patient after collecting the first data set at the first time and before collecting the second data set at the second time.

14. The method of claim 2, wherein the second time is about three months, about six months, about nine months, or about a year after the first time.

15. The method of claim 2, wherein the step of collecting the data further comprises collecting a third data set from the at least one patient at a third time, wherein the third data set indicates an ability of the at least one patient to complete a plurality of toileting tasks at the third time, wherein the third time is at least two months after the second time, and wherein the step of using the data to generate a report that describes the neurocognitive function in the at least one patient comprises indicating the progression of the neurodegenerative disease in the at least one patient based on a comparison of the first data set, the second data set, and the third data set.

16. The method of claim 15, wherein the second time is about three months, about six months, about nine months, or about a year after the first time, wherein the third time is about three months, about six months, about nine months, or about a year after the second time.

17. The method of claim 3 wherein the therapy comprises enzyme replacement, gene therapy, gene editing, a fusion protein, substrate reduction therapy, or a chaperone molecule.
